(19) United States
(12) Patent Application Publication
(10) Pub. No.: US 2015/0329827 A1
(43) Pub. Date: Nov. 19, 2015

(54) MUSE CELLS ISOLATION AND EXPANSION

(71) Applicant: RUTGERS , THE STATE UNIVERSITY OF NEW JERSEY, New Brunswick, NJ (US)

(72) Inventors: Wise Young, New Brunswick, NJ (US); Yi Ban, Piscataway, NJ (US); Dongming Sun, Princeton Junction, NJ (US); Mari Dezawa, Sendai (JP)

(21) Appl. No.: 14/654,279

(22) PCT Filed: Dec. 23, 2013
(86) PCT No.: PCT/US13/77426

§ 371 Date: Jun. 19, 2015

Related U.S. Application Data
(60) Provisional application No. 61/740,835, filed on Dec. 21, 2012.

(51) Int. Cl. C12N 5/0775

(52) U.S. Cl. CPC C12N 5/0665; C12N 2533/54; C12N 2509/00

(57) ABSTRACT

The present invention, relates to novel methods of isolating and expanding pluripotent stem cells, including multi-lineage stress enduring (MUSE) cells.

### CROSS REFERENCE TO RELATED APPLICATION

This application claims priority of U.S. Provisional Application No. 61/740,835 filed on Dec. 21, 2012. The content of the application is incorporated herein by reference in its entirety.

### FIELD OF THE INVENTION

This invention relates to a novel method of isolating and expanding pluripotent stem cells, such as multi-lineage stress enduring (MUSE) cells.

### BACKGROUND OF THE INVENTION

MUSES cells are pluripotent, non-tumorigenic stem cells, which were originally identified in adult human mesenchymal cell populations (Kuroda et all, 2010, Proceedings of the National Academy of Sciences of the United States of America 107: 8639-43). These cells are stress-tolerant and capable of self-renewing and forming characteristic cell clusters in suspension cultures. They express a set of genes associated with pluripotency and can be isolated from fibroblasts, bone marrow, or adipose tissues. They correspond to 1˜several % of cultured mesenchymal stem cells and ˜0.03% of bone marrow mononucleated cells. MUSE cells are attractive sources of autologous cells for regenerative medicine because they do not require genetic manipulation and have low tumorigenic potential (Wakao et al., 2011, Proceedings of the National Academy of Sciences of the United States of America 108: 9875-80.). However, MUSE cells are not abundant in tissues and cultured cells and those from bone marrow, fibroblast, or adipose tissue are limited in number and growth. Thus, there is a need for methods for high-yield production of MUSE cells.

### SUMMARY OF INVENTION

This invention relates to a method of enriching pluripotent stem cells, such as multi-lineage stress enduring (MUSE) cells, and related cell fractions. In one aspect, the invention provides a method of enriching pluripotent stem cells, such as MUSE cells. The method includes (i) providing a plurality of starting mesenchymal cells of an animal; (ii) plating the plurality of starting mesenchymal cells on a substrate; (iii) culturing the plurality of starting mesenchymal cells plated on the substrate in a first medium for a first period of time; and (iv) obtaining cells adherent to the substrate to produce a population of adherent mesenchymal cells. About 1% or more (e.g., 3, 4, 5, 6, or 7%) of the population of adherent mesenchymal cells are MUSE cells.

In the method, the animal can be a mammal, such as a human. The starting cells can be obtained from a living body tissue (e.g., mesodermal tissue, mesenchymal tissue, or the like of a living body) of the animal, including but not limited to umbilical cord blood, bone marrow, amniotic fluid, adipose tissue, placenta, and peripheral blood. In one embodiment, the tissue is umbilical cord blood. Preferably, the starting cells are mononuclear cells. The starting mesenchymal cells can be obtained from the animal by a method comprising osmotic gradient centrifugation.

In the above-mentioned method, the substrate can contain gelatin, collagen and poly-L-Lysine to allow cells to attach thereto. Unattached cells can be removed within about 12-36 hours (e.g. 18-30 hours or 24 hours) after the starting mesenchymal cells are plated on the substrate. The first medium can contain serum. The first period of time can be about 3-10 days (e.g., 3-5 days or 4 days). For obtaining cells adherent to the substrate, the above-described method can include detaching from the substrate (e.g., via a non-trypsin means) the cells adherent to the substrate to obtain a plurality of suspended cells.

To further purify or enrich MUSE cells, the suspended cells can be exposed to or contacted with trypsin in a second medium (e.g., a growth medium) for a second period of time (e.g., about 4-12 hours, such as 6-10 hours or 8 hours) to obtained a plurality of trypsin-exposed cells. The plurality of trypsin-exposed cells can be further cultured in suspension for a third period of time such as about 3-10 days (e.g., 4-6 days or 5 days). After the culturing in suspension step, the trypsin-exposed cells can be cultured in an adherent culture for a fourth period of time, such as about 3-10 days (e.g., 4-6 days or 5 days), to obtain an expanded cell population. About 30% or more (e.g., 35, 40, 50, 60, or 66%) of the expanded cell population are MUSE cells. To further increase the yield (the number or percentage of MUSE cells), the trypsin treatment-suspension culture-adherent culture steps can be repeated one or more times.

The invention also provides a substantially pure MUSE cell fraction/population or an enriched MUSE cell fraction/population produced according to the method described above. The invention further provides a cell fraction or an enriched cell fraction having pluripotent stem cells, such as MUSE cells, which can be produced according to the method described above. Also provided are methods of producing or enriching MUSE cells substantially as shown and described herein and MUSE cell populations substantially as shown and described herein.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a diagram showing an exemplary procedure to isolate, purify, and expand MUSE cells directly from thawed umbilical cord blood (UCB) mononuclear cells.

### DETAILED DESCRIPTION OF THE INVENTION

This invention is based, at least in part, on unexpected discoveries that MUSE cells, which are a small proportion in many tissues, can be isolated directly from some tissues (e.g., umbilical cord blood) at a much higher yield and that MUSE cells can be expanded in vitro so that a large number of MUSE cells can be produced efficiently without genetic manipulation or induction by exogenous gene or protein.

### MUSE Cells

MUSE cells are pluripotent, non-tumorigenic stem cells. These cells were originally found in adult human mesenchymal cell populations and reported in 2010 by Kuroda et al. from Mari Dezawa's laboratory at Tohoku Imperial University in Sendai, Japan. See, Kuroda et al., 2010, Proceedings of the National Academy of Sciences of the United States of America 107: 8639-43, the content of which is incorporated herein by reference in its entirety. These cells are stress-tolerant, self-renew, form characteristic cell clusters in suspension cultures, express a set of genes associated with pluripotency, and can be isolated from fibroblasts, bone marrow, or adipose tissues. Called MUSE (multi-lineage stress enduring) cells, these cells express two definitive markers: CD105 and SSEA-3. The former is a mesenchymal cell marker and the latter is a pluripotency marker expressed by human embryonic stem cells. The cells can give rise to cells of all three germ layers from a single cell, have limited growth potential until Heyflick limit, and do not form teratomas when transplanted to immunologically deficient animals.

In 2011, the Dezawa group showed that MUSE cells are likely to be the source of induced pluripotent cells (iPS) which can be generated when Yamanaka genes (Oct3/4, Sox2, Klf4 and c-Myc) are transfected in mouse or human fibroblasts. See Wakao et al., 2011, Proceedings of the National Academy of Sciences of the United States of America 108: 9875-80, the content of which is incorporated herein by reference in its entirety. More specifically, it was found that if CD105⁺/SSEA3⁺ cells were removed from fibroblasts, the Yamanaka genes did not yield any iPS cells nor did they elevate pluripotency genes after receiving Yamanaka genes. In contrast, transfection of CD105⁺/SSEA3⁺ cells from fibroblasts with the Yamanaka genes resulted in many iPS cells that form teratomas. Compared with iPS, MUSE cells are a more attractive source of autologous cells for regenerative medicine because MUSE cells do not require genetic manipulation and have low or no tumorigenic potential.

As used herein, the term MUSE cells refers to the pluripotent stem cells described in the above-mentioned Kuroda et al., 2010 and Wakao et al., 2011, as well as US Patent Application Nos. 20120244129 and 20110070647, the contents of which are incorporated herein by reference in their entireties. More specifically, MUSE cells refer to a specific type of animal (e.g., human) mesenchymal pluripotent stem cell that is capable of generating cells with the characteristics of all three germ layers from a single cell. MUSE cells are stress tolerant; morphologically indistinguishable from general mesenchymal cells in adhesion culture (resemble fibroblasts); able to form M-clusters in suspension culture that are positive for pluripotency markers and alkaline phosphatase staining; able to self-renew; not very high in their proliferation activity and not shown to form teratomas in immunodeficient mouse testes; able to differentiate into endodermal, ectodermal, and mesodermal cells both in vitro and in vivo; and positive for both CD105 and SSEA-3.

MUSE cells may also express pluripotency markers such as Nanog, Oct3/4, and Sox2, and are negative for NG2 (a marker for perivascular cells), CD34 (a marker for endothelial progenitors and adipose-derived stem cells), von Willebrand factor (a marker for endothelial progenitors), CD31 (a marker for endothelial progenitors), CD117 (c-kit, a marker for melanoblasts), CD146 (a marker for perivascular cells and adipose-derived stem cells), CD271 (a marker for neural crest-derived stem cells), Sox10 (a marker for neural crest-derived stem cells), Snail (a marker for skin-derived precursors), Slug (a marker for skin-derived precursors), Tyrp1 (a marker for melanoblasts), and Dct (a marker for melanoblasts) by flow cytometry analysis or by RT-PCR.

As used herein, the phrase “negative for” a marker or an antigen refers to a situation in which, when FACS (fluorescence activated cell sorting) analysis is conducted as described below, cells are not sorted as positive cells or when expression is examined by RT-PCR, no expression thereof is confirmed. That is, even if such a marker or an antigen is expressed to a degree such that it is undetectable by such techniques, cells are designated as negative in the present invention. Alternatively, the phrase “negative for” a marker or an antigen refers to a situation where measurement of the marker or antigen is performed with positive control cells known to be positive for the marker or antigen or a negative control cells known to be negative for the marker or antigen. When almost no expression is detected, or the expression level is significantly lower compared with such positive control cells, or the expression level is statistically no different from such negative positive control cells, cells may be designated as negative.

MUSE cells from bone marrow, fibroblast, or adipose tissue are limited in number and growth capacity. The cells are not abundant in bone marrow aspirates and about only 1:3,000 of bone marrow mononucleated cells are MUSE cells. In cultured mesenchymal cells, MUSE cells account for only several percentages of fibroblasts and bone marrow stromal cells. Once isolated and cultured in suspension, MUSE cells typically grow for only several weeks and then cease proliferation but after transferring to adherent culture, they start proliferation. Accordingly, merely isolating CD105⁺SSEA3⁺ cells from marrow mononucleated cells and subsequent conventional culturing such isolated cells may not provide sufficient MUSE cells for practical uses.

Even though MUSE cells have limited proliferation in suspension cultures, they keep on growing until their Hayflick limit in adherent culture. This limit is 40-60 divisions in human fetal cell cultures. In cultures of older adult cells, depending on the age of the cells, the Hayflick limit should be less. Umbilical cord blood cells, being the youngest post-natal source of cells, should have more proliferation capacity. Similar to other somatic stem cells and hematopoietic stem cells. MUSE cells generate themselves by symmetric cell division but, at the same time, randomly produce non-MUSE cells by asymmetric cell division. Therefore, initially purified MUSE cell cultures show a sigmoidal decline in their concentration in culture, reaching at plateau of several percent, and then maintain this lower concentration. Yet, as disclosed herein, the method of this invention allows one to increase the concentration of MUSE cells in vitro.

### Umbilical Cord Blood

Umbilical cord blood contains a high proportion of stem cells and progenitor cells. These include CD34⁺ endothelial precursor cells, CD133⁺ pluripotent stem cells, and other progenitor cells. In the inventors' experience, as much as 0.3% of mononuclear cells isolated by density centrifugation from frozen umbilical cord blood units are CD34⁺ or CD133⁺. The latter cells are pluripotent. While CD34⁺ cells can be grown in culture, the growth must be stimulated by cytokines, including Steel factor (SF) and interleukin-6. Seligman et al. (Stem Cells and Development 2009, 18: 1263-71) has described a method of isolating pluripotent or multipotent stem cells from blood. Some investigators have described procedures for growing neuron-like cells from umbilical cord blood mesenchymal cells. Most of these cells are CD133⁺. Others have described methods of growing neural progenitors, neurons, and oligodendroglial cells from umbilical cord blood, as well as cardiomyocytes and hematopoietic stem cells.

To the effective filing date of this application, nobody had reported discovery of CD105⁺/SSEA3⁺ cells in umbilical cord blood. As disclosed herein, assays were carried out to look for such cells in mononuclear cells isolated from thawed human umbilical cord blood units. Using the Miltenyi flow cytometer, it was unexpectedly found that an average of 0.8% of mononuclear cells is both CD105⁺ and SSEA3⁺. This concentration is about 1000 times higher than that in bone marrow, fibroblasts, or adipose tissues. The high incidence of MUSE cells in umbilical cord blood may explain why many investigators have reported much higher efficiency of iPS generation in umbilical cord blood cells.

Umbilical cord blood thus is one of the richest sources of MUSE cells so far. Many cord blood banks around the world store hundreds of thousands of cord blood units. Unlike bone marrow, 80% of umbilical cord blood units will engraft despite only 4:6 HLA match. MUSE cells from umbilical cord blood are therefore an HLA-matchable source of pluripotent stem cells for regenerative medicine. However, if 0.8% of umbilical cord blood mononuclear cells are MUSE cells, a single unit of umbilical cord blood containing 100 million cells should contain less than a million MUSE cells. Even if there were a method of harvesting all the MUSE cells from umbilical cord blood, a million MUSE cells may still not be sufficient for treatment purposes.

Currently available methods for isolating MUSE cells from tissue such as umbilical cord blood use negative lineage selection and then laser sorting of the cells. These conventional methods are not only inefficient but also expensive. Negative lineage selection and then laser sorting of the cells utilizes a great deal of antibodies. Furthermore, umbilical cord blood has many other colony forming cells. Therefore, it is difficult to grow a large population of pure MUSE cells and multiple selection procedures will be necessary before and after expanding the cells. The novel method described herein can allow one to simultaneously isolate and expand a single unit of umbilical cord blood to yield many millions of MUSE cells without use of antibodies or other expensive reagents.

### Isolating and Expanding MUSE Cells

As mentioned above, various conventional ways have been used to isolate MUSE cells. For example, MUSE cells can be isolated by first negatively selecting cells that express standard lineage markers (using CD5, CD45R, CD11b, Anti-Gr-1, 7-4, and Ter-119 antibodies) and then using laser sorting to select Lin-cells that express both CD105 and SSEA3. Another way of isolating the cells is to use magnetic nanobeads that bind to cells expressing CD105 or SSEA3 and then passing the cells through magnetized columns. Yet, these conventional methods are expensive due to uses of various antibodies and do not have high yield.

The invention provides a novel method of isolating and expanding pluripotent MUSE cells directly from umbilical cord blood and other tissues. The method does not require antibody selection of cells and allows one to expand MUSE cells in vitro and to obtain a large quantity of MUSE cells.

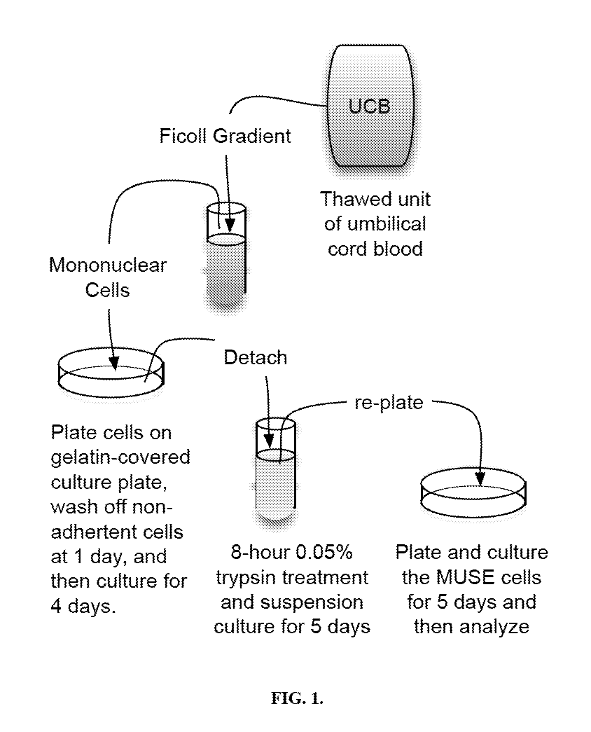

FIG. 1 shows a diagram of an exemplary procedure to isolate, purify, and expand MUSE cells directly from thawed umbilical cord blood mononuclear cells. In this exemplary procedure, either plasma-depleted or red cell reduced frozen units are thawed. Mononuclear cells are isolated by centrifuging the cells in Ficoll gradient, plated on gelatin-coated culture dishes, washed at 24 hours to remove non-adherent cells, and then cultured for 4 days. The cells are detached with a non-trypsin cell detachment solution and placed into a suspension medium. Samples of the cells can then be removed for flow cytometry analysis and the remainder is exposed to 0.05% trypsin solution for 8 hours. The cells are then grown in suspension medium for 5 days and then in adherent cultures for 5 days and reanalyzed by flow cytometry. One example of the method includes the following steps:

1. Isolation of Umbilical Cord Blood Mononuclear Cells.

Using fresh or thawed umbilical cord blood, mononuclear cells are isolated by osmotic gradient (Ficoll) to obtain the buffy-coat layer from either plasma-depleted or red cell reduced cord blood units. The procedure to isolate cells from plasma-depleted cord blood units is known in the art and typically yields about 1 million mononuclear cells per ml, up to 100 million mononuclear cells per unit of umbilical cord blood.

2. Isolation of Adherent Mesenchymal Cells.

The mononuclear cells are plated on gelatin-coated plates and grown in Minimum Essential Medium (MEM) alpha modification, containing 10% fetal bovine serum (FBS) or human cord blood serum and 0.8% MC4100. Cells that do not attach are washed away with a media change after 24 hours and the cells are then cultured for 3-5 days. At the end of 5 days, close to 100% of the adherent cells should be CD105⁺.

3. Purification of MUSE Cells.

The adherent cells are detached with a non-trypsin containing cell detachment solution, suspended and analyzed with a flow cytometer. At that time about 6-7% of the cells should be both CD105⁺ and SSEA3⁺. The suspended cells are exposed to trypsin (0.05%) for 8 hours, washed, and re-suspended in growth media. The trypsin should kill most of the non-MUSE mesenchymal cells while MUSE cells should proliferate in suspension culture. The duration trypsin exposure can be varied and repeated, e.g. 3 hours of 0.05% trypsin media followed by 2 hours in non-trypsin media and then 3 hours in 0.05% trypsin media.

4. Expansion of MUSE Cells.

The trypsin-exposed cells are grown in suspension for 5 days and then grown for another 5 days in adherent culture. At the end of this 10-day growth period, over 60% of the cells should be both CD105⁺ and SSEA3⁺. Starting from about 40 million umbilical cord mononuclear cells, one ends up with about 9 million cells of which 66% were CD105 ⁺ and SSEA3⁺. The cells can also be grown directly in adherent culture after exposure to trypsin skipping the step in suspension culture. Muse cells from non-blood tissues may not grow as well in suspension culture.

This procedure is based on three characteristics of MUSE cells. First, MUSE cells are mesenchymal cells that attach and grow in adherent cultures. By growing the cells initially in gelatin-coated culture plates and washing away all non-attached cells, the procedure rapidly and efficiently eliminates most non-mesenchymal cells. The data disclosed herein indicates that this procedure eliminates nearly all cells that do not express CD105, a marker of mesenchymal cells. This step unexpectedly results in an eight-fold enrichment for MUSE cells, from about 0.8% to about 6-7%. Second, MUSE cells are stress-tolerant. For example, the cells can survive long periods of trypsin treatment. Third, MUSE cells proliferate in suspension culture. Fibroblasts and other differentiated cells do not proliferate in suspension. This further enriches and purifies the MUSE cells. The data discussed herein suggest that exposure to 8 hour of 0.05% trypsin results, followed by growth in suspended and then adherent culture results in a ten-fold enrichment for MUSE cells from about 6-7% to over 60%. Note that the third and fourth steps of the procedure can be repeated to further increase the number and percentage of MUSE cells.

In the above-described procedure, the concentration of trypsin and the trypsin exposure time duration are exemplary and not limited. For example, in general culture of adherent cells, trypsin may be used at concentrations ranging from 0.1% to 1%, e.g., 0.1% to 0.5%, for various time durations for removal of adherent cells adhering to a culture vessel. Here, cells similarly can be exposed to a trypsin solution with a higher trypsin concentration for shorter time duration, or a trypsin solution with a lower trypsin concentration for longer time duration. The time for trypsin incubation can range from about 3 to 24 hours. One skilled in the art could determine the suitable trypsin concentration and time duration in view of the disclosure herein.

Regarding the medium to be used for culturing cells from mesodermal tissue, mesenchymal tissue, or the like of a living body and culture conditions, any medium and culture conditions generally used for culturing animal cells may be employed. Also, a known medium for culturing stem cells may be used. A medium may be appropriately supplemented with serum such as fetal calf serum, human umbilical cord blood serum, antibiotics such as penicillin and streptomycin, and various bioactive substances.

Compared with conventional antibody-based methods for sorting to enrich MUSE cell concentrations, the procedure disclosed herein is much more efficient. It is also an inexpensive way of isolating and expanding large numbers of MUSE cells. Other methods not only cannot yield millions of cells but require expensive reagents (e.g., antibodies) and instruments (such as FACS sorters). The procedure disclosed herein does not require antibodies for positive or negative selection, laser sorting, or other expensive reagents or instruments.

The method can be used to isolate, purify, and expand MUSE cells directly from umbilical cord blood or any source of MUSE cells. Umbilical cord blood is attractive source of MUSE cells for the following reasons. First, HLA-matched umbilical cord blood is a rich and immune-compatible source of MUSE cells. Many umbilical cord blood banks have stored hundreds of thousands of cord blood units that can be HLA-matched to provide immune-compatible MUSE stem cells for transplantation purposes. Second umbilical cord blood cells have greater expansion potential than other sources of adult mesenchymal stem cells obtained from bone marrow, skin, or fat. Third, umbilical cord blood has a long history of safe use in bone marrow replacement with a low tumorigenesis risk.

This method disclosed herein is applicable for isolating and expanding MUSE cells from other tissues besides umbilical cord blood. As mentioned above, MUSE cells are a special subpopulation of pluripotent stem cells isolated from mesenchymal stem cells. Thus, any sources suitable for isolating mesenchymal stem cells can be used to practice the invention disclosed herein. Examples include umbilical cord blood, umbilical cord, umbilical cord stroma cells (Wharton's jelly), amniotic membranes, placenta, umbilical cord lining, and even menstrual blood. Other examples include bone marrow, skin, adipose tissues, and even peripheral blood. However, as pointed out above, none of these sources have as many MUSE cells and may have less growth potential than umbilical cord blood cells.

Because mesenchymal stem cells contain MUSE cells, many beneficial effects and pluripotency or neural tendency of mesenchymal stem cells may have been due to the MUSE cells amongst mesenchymal cells. Although there have been many descriptions of methods to grow mesenchymal stem cells from these sources, none have specifically focused on isolating and expanding MUSE cells from these sources and particularly not from umbilical cord blood.

The method disclosed in this invention allows one to isolate, enrich, expand, or obtain pluripotent stem cells, such as MUSE cells, directly from various tissues including umbilical cord blood. The invention further provides a cell fraction or an enriched cell fraction having pluripotent stem cells, such as MUSE cells, which can be produced according to the method described above.

As used herein, the phrase that one can “directly” isolate, enrich, expand, or obtain pluripotent stem cells from a tissue means that cells can be isolated from tissue without any artificial induction/genetic reprogramming operation such as introduction of a reprogramming foreign/exogenous gene or protein, or treatment with a compound (e.g., administration of a compound). Such foreign gene may be, but is not limited to, a gene capable of reprogramming the nucleus of a somatic cell. Examples of such foreign gene include Oct family genes such as an Oct3/4 gene, Klf family genes such as a Klf gene, Myc family genes such as a c-Myc gene, and Sox family genes such as a Sox2 gene. Also, examples of a foreign protein include proteins encoded by these genes and cytokines. Furthermore, examples of a compound include a low-molecular-weight compound capable of inducing the expression of the above gene that can reprogram the nucleus of a somatic cell, DMSO, a compound that can function as a reducing agent, and a DNA methylating agent.

In addition, in the present invention, routine cell culturing and passing (e.g., that using trypsin), isolation of a cell or a cell fraction using a cell surface marker as an index, exposure of cells to cellular stress, and provision of a physical impact on cells are not artificial induction operation mentioned above. Accordingly, the pluripotent stem cells of the present invention may also be characterized in that they can be obtained without requiring reprogramming or induction of dedifferentiation.

In addition, the method disclosed in this invention allows one to isolate, enrich, expand, or obtain pluripotent stem cells, such as MUSE cells, directly from various tissues without using antibody specific for a cell marker either for positive selecting (e.g., one or more of SSEA-3 and CD105) or for negative selecting (e.g., one or more of CD5, CD45R, CD11b, Anti-Gr-1, 7-4, and Ter-119). Accordingly, in an embodiment, the invention also provides methods for isolating, enriching, expanding, or obtaining pluripotent stem cells, such as MUSE cells, directly from various tissues without using antibody specific for a cell marker.

The pluripotent stem cells of the present invention are present in mesodermal tissue or mesenchymal tissue, or the like of a living body. In the present invention, cells or cell fractions existing in these types of tissue are isolated. As used herein “pluripotent stem cell” refers to a cell having the ability to give rise to cell types of all three embryonic germ layers, namely endodermal, mesodermal, and ectodermal cells from a single cells, and that having the ability to self-renew. In preferred embodiments, the pluripotent stem cells of this invention have the following properties. The pluripotent stem cells express pluripotency markers such as Nanog, Oct3/4, SSEA-3, PAR-4, and Sox2. The pluripotent stem cells exhibit clonality by which they expand from a single cell and keep producing clones of themselves. The pluripotent stem cells exhibit self-renewal capability. The pluripotent stem cells can differentiate in vitro and in vivo into the three germ layers (i.e., endodermal cell lineage, mesodermal cell lineage, and ectodermal cell lineage). The pluripotent stem cells differentiate into the three germ layers when transplanted into the testis or subcutaneous tissue of a mouse. The pluripotent stem cells are found to be positive through alkaline phosphatase staining.

The pluripotent stem cells of the present invention are clearly distinguished from adult stem cells and tissue stem cells in that pluripotent stem cells of the present invention are pluripotent and have greater differential potential. Also, the pluripotent stem cells of the present invention are clearly distinguished from cell fractions such as bone marrow stromal cells (MSC) in that pluripotent stem cells of the present invention are isolated in the form of a single cell or a plurality of cells having pluripotency. The pluripotent stem cells of the present invention are clearly distinguished from iPS cells (induced pluripotent stem cells) and ES cells in that the pluripotent stem cells of the present invention can be directly obtained from living bodies or tissue.

Moreover, the pluripotent stem cells of the present invention have the following properties. (i) The growth rate is relatively slow and the division cycle takes 1 day or more, such as 1.2-1.5 days. However, the pluripotent stem cells do not exhibit infinite proliferation in a manner similar to ES cells or iPS cells. (ii) When transplanted into an immunodeficient mouse, the pluripotent stem cells differentiate into an endodermal cell lineage, a mesodermal cell lineage, and an ectodermal cell lineage. The pluripotent stem cells have not been observed to become tumorigenic cells, unlike ES cells or iPS cells, which usually form teratomas within short time periods, such as 8 weeks. (iii) The pluripotent stem cells form ES cell-derived embryoid body-like cell clusters as a result of suspension culture. (iv) The pluripotent stem cells form embryoid body-like cell clusters as a result of suspension culture and stop growth within about 10-14 days. Subsequently, when the clusters are transferred for adherent culture, they start to grow again. (v) Asymmetric division is associated with growth. (vi) The karyotypes of the cells are normal. (vii) The pluripotent stem cells have no or low telomerase activity. (viii) Regarding methylation state, methylation levels in Nanog and Oct3/4 promoter regions are low in iPS cells induced from the pluripotent stem cells of this invention, e.g., MUSE cells. (ix) The pluripotent stem cells exhibit high phagocytic ability. (x) The pluripotent stem cells exhibit no tumorigenic proliferation.

As used herein the phase “have no or low telomerase activity” refers to no or low telomerase activity being detected when such activity is detected using a TRAPEZE XL telomerase detection kit (Millipore), for example. The term “low telomerase activity” refers to a situation in which cells have telomerase activity to the same degree as that of human fibroblasts or have telomerase activity that is ⅕ or less and preferably 1/10 or less that of Hela cells.

The expression “cells exhibit no tumorigenic proliferation” as used herein refers to a situation in which, when suspension culture is performed, the cells stop their growth at the time when their clusters reach a predetermined size and do not undergo infinite growth. Moreover, such expression refers to a situation in which, when such cells are transplanted into the testis of an immunodeficient mouse, no teratoma is formed. In addition, the above (i) to (iv) and the like also relate to the fact that the relevant cells (clusters) do not undergo tumorigenic proliferation.

The pluripotent stem cells of the present invention (e.g., MUSE cells) are capable of differentiating into the three germ layers through in vitro adherent culture. Specifically, the pluripotent stem cells can differentiate into cells representative of the three germ layers, including skin, liver, nerve, muscle, bone, fat, and the like through in vitro induction culture. Also, the pluripotent stem cells are capable of differentiating into cells characteristic of the three germ layers when transplanted in vivo; the pluripotent stem cells are capable of surviving and differentiating into organs (e.g., skin, spinal cord, liver, and muscle) when transplanted to the damaged organs via intravenous injection into a living body.

Accordingly, once the above-described pluripotent stem cells, such as MUSE cells, are isolated or enriched, the cells can be then tested by standard techniques to confirm the differentiation potential of the cells using one or more of lineage-specific markers. That is, one can test whether, under suitable culturing conditions, the cells can be induced to differentiate and give rise cells expressing markers for the three germ layers. Exemplary markers for ectodermal cells include nestin, NeuroD, Musashi, neurofilament, MAP-2, and melanocyte markers (such as tyrosinase, MITF, gf100, TRP-1, and DCT); exemplary markers for mesodermal cells include brachyury, Nkx2-5 smooth muscle actin, osteocalein, oil red-(+) lipid droplets, and desmin; exemplary markers for endodermal cells include GALA-6, α-fetoprotein, cytokeratin-7, and albumin.

For example, isolated/enriched cells can be induced to form neuro-glial cells, osteocyte, and adipocyte by methods known in the art. Briefly the cells can be passed and cultured to confluence, shifted to an osteogenic medium or an adipogenic medium, and incubated for suitable time (e.g., 3 weeks). The differentiation potential for osteogenesis can be assessed by the mineralization of calcium accumulation, which can be visualized by von Kossa staining. To examine adipogenic differentiation, intracellular lipid droplets can be stained by Oil Red O and observed under a microscope. For neural differentiation, the cells can be incubated in a neurogenic medium for suitable duration (e.g., 7 days), and then subjected to serum depletion and incubation of β-mercaptoethanol. After differentiation, cells exhibit the morphology of retractile cell body with extended neuritelike structures arranged into a network. Immunocytochemical stain of lineage specific markers can be further conducted to confirm neural differentiation. Examples of the markers include neuron specific class III β-tubulin (Tuj-1), neurofilament, and GFAP.

Once pluripotent stem cells, such as MUSE cells, are isolated and their differentiation potential confirmed, the cells or cell populations prepared by the methods described above can be used in a variety of ways. Due to their pluripotency and non-tumorigenicity, the cells or cell populations can be used for treating various degenerative or inherited diseases, while avoiding ethical considerations of human embryo manipulation and tumorigenic risks associated with other stem cells such as ES cells and iPS cells. Furthermore, since the method of this invention allows one to obtain a large quantity of pluripotent stem cells, such as MUSE cells, one can also avoid logistical obstacles associated with other types of stem cells.

In one example, one can use the cells for treating various conditions, including spinal cord injury, demyelination conditions, traumatic brain injury and stroke, as well as suppressing unwanted immune responses (e.g., inflammation) and treating disorders of heart, lung, gut, liver, pancreas, muscle, bone marrow, and skin. To that end, one can test the cells for pluripotency first in vitro and then in vivo, and then in uninjured immune-deficient animals, and finally in spinal-injured animals and other models of central nervous system and other tissue damage.

As used herein, the term “cell fraction” refers to a cell population containing at least a given amount of a desired cell (e.g., MUSE cell). The term “pluripotent stem cell fraction” refers to a cell population containing a pluripotent stem cell in an amount corresponding to 1%, 2%, 3%, 6% or more thereof, 10% or more thereof, 30% or more thereof, 50% or more thereof, 70% or more thereof, 90% or more thereof, or 95% or more thereof. Examples thereof include cell clusters obtained via culture of pluripotent stem cells and cell populations obtained via enrichment of pluripotent stem cells. Also, the cell fraction may also be referred to as a substantially homogenous cell fraction.

The term “living body” as used herein refers to a living animal (e.g., mammalian) body, and it specifically refers to an animal body that undergoes development to some extent. In the present invention, examples of such living body do not include fertilized eggs or embryos at development stages before the blastula stage, but include embryos at development stages on and after the blastula stage, such as fetuses and blastulae. Examples of mammals include, but are not limited to, primates such as humans and monkeys, rodents such as mice, rats, rabbits, and guinea pigs, cats, dogs, sheep, pigs, cattle, horses, donkeys, goats, and ferrets. The pluripotent stem cells of the present invention, e.g., MUSE cells, are distinguished from embryonic stem cells (ES cells) or embryonic germ stem cells (EG cells) in that they are from living body tissue.

The term “mesodermal tissue” refers to tissue of mesodermal origin that appears in the course of initial development of an animal. Examples of mesodermal tissue include tissue of the muscular system, connective tissue, tissue of the circulatory system, tissue of the excretory system, and tissue of the genital system. For example, the pluripotent stem cells of the present invention can be obtained from bone marrow aspirates or skin tissue such as dermal connective tissue. The term “mesenchymal tissue” refers to tissue such as bone, cartilage, fat, blood, bone marrow, skeletal muscle, dermis, ligament, tendon, dental pulp and umbilical cord. For example, the pluripotent stem cells of the present invention can be obtained from umbilical cord, bone marrow or skin.

Examples of mesodermal tissue and mesenchymal tissue of a living body include, but are not limited to, bone-marrow mononuclear cells, fibroblast fractions such as skin cells, pulp tissue, eyeball tissue, and hair root tissue. As cells, both cultured cells and cells collected from tissue can be used. Among these cells, umbilical cord cells, bone marrow cells and skin cells are preferred. Examples of such cells include a human bone marrow stromal cell (MSC) fraction and a human dermal fibroblast fraction. A bone MSC fraction can be obtained by culturing a bone marrow aspirate for 2 to 3 weeks.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, “about 1” may mean from 0.9-1.1, and “about 4” may mean from 3.6-4.4. Other meanings of “about” may be apparent from the context, such as rounding off, so, for example “about 1” may also mean from 0.5 to 1.4.

### EXAMPLES

In the following examples, the method as shown in FIG. 1 was carried out to isolate and expand MUSE cells directly. The method does not require antibody selection of cells and was applied to a red cell reduced (RCR) unit of umbilical cord blood obtained from Stemcyte, Inc.

### Example 1

This example describes methods used in EXAMPLES 2 and 3 below.

Isolation of Monomuclear Cells

Mononuclear cells were isolated from umbilical cord blood by centrifugation in a Ficoll gradient, resulting in a buffy coat layer that contained the mononuclear cells. When cells were isolated from plasma depleted (PD) frozen units, osmotic shock was used to reduce the number of red blood cells and DNAase to prevent sticking of the cells to each other in the Ficoll gradient. PD usually yielded about a million cells per ml of thawed cord blood while thawed RCR units were 25 ml and contained 200-250 million mononuclear cells. About 40 million mononuclear cells were used.

Culturing

The cells were plated for 24 hours on gelatin-coated or other culture dishes to which mesenchymal cells attach and the non-adherent cells were washed off with phosphate-buffered saline. The cells were then in adherent culture in Minimal Essential Medium Eagle (MEM) Alpha Modifications, 10% FBS, and 0.8% MC4100. MEM alpha modification is a synthetic culture medium modified to have higher amino acid concentrations, Earle's balanced salts, non-essential amino acids, sodium pyruvate, and vitamins (See, www.safeglobal.com/etc/medialib/docs/Sigma/Formulation/m0894for.Par.0001.File.tmp/m0894for.pdf).

At the end of four days, the cells were detached with a non-trypsin cell detachment solution, Accutase Cell Detachment Solution, containing proteolytic and collagenase enzymes. The solution did not contain trypsin or EDTA. A sample was removed for analysis by flow cytometer. The remaining, re-suspended cells were incubated in 0.05% trypsin for 8 hours and allowed to proliferate in suspension for 5 days, re-plated onto gelatin coated plates, allowed to grow for 5 days, and then analyzed by flow cytometry.

Flow Cytometry

A Miltenyi MACSQuant Analyzer flow cytometer was used for this study. For all flow cytometry readings, living cells were identified on scatterplots of propidium iodide (PI)/PE-Cy5,5-A and PE-A. Propidium iodide is a fluorescent dye that intercalates into double-stranded nucleic acid. It is normally excluded from viable cells but will penetrate membranes of dead and dying cells. When excited by 488 nm laser, PI emission can be detected in the red fluorescence channel.

Dead cells showed progressive increase of PI in a 45° “tail” and were excluded from the analysis below. To identify single or double markers on the cells, the cells were incubated with primary antibodies specific for CD105, SSEA3, CD34, and CD45. Secondary fluorescent antibodies labeled with either Allophycocyanin (APC-A) or fluorescen isothyocynate (FITC) were then added. Allophycocyanin has an excitation wavelength of 650 nm, an emission wavelength of 660 nm, (red), and a molecular weight of 104K; fluorescen isothyocynate has an excitation wavelength of 495 nm, an emission wavelength of 519 nm (green), and a molecular weight of 389. Then non-specific fluorescence was normalized out by using a non-specific isotype antibody, setting the lower boundaries for detection of positive cells. Fluorescence compensation was used to exclude spectral overlap when two channels of fluorescence were used.

### Example 2

Mononuclear cells were isolated from a thawed unit of red cell reduced cord blood unit by Ficoll gradient centrifugation. The cells were then plated on gelatin-coated cell culture dishes, washed with phosphate buffered saline at 24 hours to remove non-adherent cells, and then cultured for 4 more days. After 5 days of adherent culture on gelatin-coated dishes, the umbilical cord blood mononuclear cells were then detached using a cell detachment solution and analyzed the cells by flow cytometry. It was found that after 5 days of adherent culture almost all the mononuclear cells expressed CD105, a mesenchymal cell marker.

The resulting flow cytometry scatterplots showed a variety of cells ranging from very low to very high side scatter and forward scatter. Application of propidium iodide (PI) and a phycoerythrin marker PE-Cy5,5-A to the cells showed a linear 45° “tail” of cells on scatterplots of PI/PE vs. PE. Since PI only gets into dead or dying cells, cells resulting this tail of increasing PI that correspond to PE were likely to be dead and therefore excluded from the further analysis.

Of the remaining cells, nearly all (99.2%) express CD105. To that end, flow cytometry scatterplots were obtained. The plots indicating flow-cytometry data on two sets of cells were obtained from the same source.

In one set of scatterplots, three scatterplots were analyses of cells incubated with a control isotype primary antibody that does not bind CD105; another three scatterplots were analyses of the cells incubated with a CD105 antibody and a secondary fluorescent antibody. Among them, two graphs were scatterplots of side-scatter (SSC-A Y-axis) and forward scatter (FSC-A X-axis). The cells could be seen to have similar scatter in the isotype-control and CD105-labeled cells. The scatterplots included two other scatterplots of the ratio of PI/PE-Cy5,5A (Y-axis) and PE-A. PI refers to propidium iodide that enters dead cells and intercalates among the DNA. On each of these two scatterplots, a tail of cells angled 45° to the upper right. Dead cells were located in this tail, indicating an increase in propidium iodide. The last two graphs show scatterplots of APC-A signal conjugated to the isotype (ISO) or to the CD105 antibody. One of the scatterplot shows all the cells located on the lower left (low side-scatter and low APC-A signal). The exclusion line was set to exclude all cells that express APC-A signal in the range of the isotype control. The other scatterplot showed the location of the cells in the lower right side of the graph. The data shows that 99.2% of the labeled cells were CD105⁺.

The distribution of cells labeled with the isotype control and the CD105-APC-A antibody were compared. There was very little overlap between the two populations, as shown in signal intensity histograms. To that end, scatterplots of side-scatter (SSC-A) and of cells incubated with the isotype control antibody (ISO-APC-A) and cells incubated with the CD105 antibody (CD105-APC-A) were obtained. Also obtained were related histograms showing cell distribution at each signal intensity category. There was almost no overlap between the two populations of cells.

CD90 expression in the mononuclear cells after 5 days of adherent culture was also examined and a set of diagrams showing CD90 expression obtained. Two of the obtained graphs showed scatterplots of cells incubated with control isotype (ISOCD90-PE-A) and CD90 antibody (CD90-PE-A). Another graph showed a histogram of the same results, showing some overlap of the two groups. Analysis indicated that 96.59% of the cells expressed CD90.

These results indicate that nearly all (96.59%) of the cultured adherent cells also expressed CD90. CD-90 is Thy-1, a GPI-linked surface glycoprotein that is a member of the immunoglobulin superfamily and expressed by a subset of CD34⁺ hematopoietic stem cells capable of long-term growth in culture. Bone marrow stromal and fibroblast cell lines, activated endothelium, and tumor cell lines of neuronal and lymphoid origin express CD-90. The molecule plays a role in cell adhesion and migration. It was recently showed that these cells have fibroblast morphology, have a doubling time of 24.15±0.49 hours, express Nanog, Oct-4, and CD105, and have a high expansion potential (i.e. 10¹⁰ cells in 30 days).

CD34 expression was also examined in the cells after 5 days of adherent culture and a set of diagrams were obtained. It was found that adherent cultures were CD34 negative. Two of the scatterplots showed respectively the distribution of cells incubated with the control isotype antibody and the distribution of cells incubated with the CD34 antibody (CD34-FITC-H). It was found that few or no (0.0% and 0.22%) cells expressed CD34.

These results indicate that the above-obtained adherent mesenchymal cells did not express CD34 as there was no difference in fluorescence of cells labeled with the isotype control and the CD34 antibody. A marker of endothelia progenitor cells, CD34⁺ is the most commonly used surrogate marker for hematopoietic stem cells in umbilical cord blood. Some hematopoietic stem cells express CD34. The above data showed clearly that the CD105-positive cells that were cultured on gelatin coated culture dishes do not express CD34 and that less than 0.22% of the cells isolated by adherent growth on gelatin-coated plates express CD34.

Assays were then carried out to examine CD45 expression after 5 days of adherent culture. Diagrams of scatterplots showing CD45 expression after 5 days of adherent culture were obtained. It was found that some of the adherent mesenchymal cell expressed CD45. In two experiments, 10.37% and 27.26% of the adherent mesenchymal cells were found to express CD45. The CD45 family of glycoprotein belongs to a family of protein tyrosine phosphatase receptor type C (PTPRC). Present on all differentiated hematopoietic cells, except erythrocytes and plasma cells, CD45 is expressed by naive lymphocytes, lymphomas, chronic lymphocytic leukemia, and acute non-lymphocytic leukemia cells. CD45 is commonly used to distinguish lymphomas from carcinomas.

Assays were also carried out to examine CD105 and SSEA3 after 5 days of adherent culture of the cells. Briefly, the cells were incubated with antibodies for CD105 and SSEA3 or two control antibodies (for CD105 and SSEA3). It was found that the fluorescent signals for both control antibodies were low. In contract, a scatterplot of cells incubated in both antibodies for CD105 and SSEA3 showed that only 6.93% of all living cells were CD105⁺ and SSEA3⁺ and a small percentage of the cells (1.17%) did not express SSEA3 or CD105. There was substantial overlap between the control isotype antibody and SSEA3 antibody.

These results indicated that about 6-7% of the CD105 expressing adherent cells also expressed SSEA3, the human embyronic stem marker identified by Dezawa, et al. Although there is some overlap between the control isotype antibody and the SSEA3 antibody, the data suggests that about 6-7% of the CD105⁺ cells express SSEA3. A small percentage (1.17%) of the cells are negative for both CD105 and SSEA3. In previous studies, it was shown that about 0.8% of mononuclear cells isolated from frozen cord blood units express both CD105 and SSEA3. Therefore, this suggests that the first step of growing the cells in gelatin-coated dishes surprisingly enriched the cultures by nearly tenfold from about 0.8% to about 6-7%.

### Example 3

The adherent mesenchymal cells obtained in the manner described in EXAMPLE 2 above were then exposed to 0.05% trypsin for 8 hours, cultured for 5 days in suspension cultures and then 5 days in adherent cultures. At the end of 10 days of culturing, the cells were detached with a non-trypsin detachment solution. Then, the above-described assays were performed to examine various markers.

CD105 expression was examined and related scatterplots were obtained. The obtained scatterplots included a graph showing a scatterplot of the cells distributed by side scatter (SSC-A) and forward scatter (FSC-A), and a graph of the PI/PE-Cy5,5A vs. PE-A distribution, showing a 45° “tail” of about 13% dead cells, which were eliminated from further analysis. The obtained scatterplots included a graph showing the isotype control and one showing the cells expressing CD105. It was found that almost all the cells (99.41%) express CD105, after eliminating the dead cells (i.e. high PI/PE ratio) from the analysis.

However, unlike the earlier samples in EXAMPLE 2 above that had been subjected to only 5 days growth in adherent culture without trypsin treatment, trypsin-treated adherent mesenchymal cells that were grown in suspension and then again in adherent cultures for ten days had few or no cells expressing CD45. In fact, the related scatterplots and histograms from two separate experiments showed that few cells express CD45 (0.08-1.83%).

Next, SSEA3 expression was examined and related scatterplots were obtained in the manner described above. Labelling the cells for SSEA3 indicated that 66.23% of the adherent mesenchymal cells expressed SSEA3. Since nearly all the cells are CD105⁺, this finding indicated that the second part of the procedure (i.e. treatment with trypsin and growth in suspension and then adherent cultures) enriched MUSE cells in culture by about another tenfold (i.e. from about 6-7% to 66%).

The related scatterplots and histogram were also obtained for cells labeled with control isotype (ISO-FITC-A) and SSEA3 antibody (SSEA3-FITC-A). The results indicated that some overlap between the control isotype and SSEA3 antibody but over two thirds (66%) of the cells clearly express SSEA3 (SSEA3 positive).

Then, CD105, CD73, and CD90 expressions were examined in the manner described above. The related scatterplots showed that almost all the cells (99.48%) were CD105⁺. It was also found that only 12.63% of the cells expressed CD73 but 96.59% expressed CD90.

The morphology of the cells obtained in this example was examined and photographs taken using phase contrast photomicrographs of living cultures at 63×. It was found that the cells resembled those of fibroblasts. The adherent mesenchymal cells were typically spindle shaped, bipolar cells. These findings are consistent with other investigators (Zhang el al, (2012), Cell Biochemistry And Function, Volume 30, Issue 8, pages 643-649,) who have described colonies of fibroblast-like cells that can be easily isolated by a single enzymatic digestion and that express CD73, CD90, and CD105 but not CD34, CD45, or HLA-DR. When cultured in differentiation media, a variety of different cells were seen. These cells included adipocytes, osteocytes, and chondrocytes. On the other hand, these are clearly mesenchymal stem cells whereas the cells obtained in this example were CD105⁺ and SSEA3⁺ MUSE cells.

In summary, mononuclear cells isolated from human umbilical cord blood can be enriched to obtain substantially pure mesenchymal cells by adherent culture on gelatin-coated plates. The percentage of MUSE (CD105⁺ and SSEA3⁺) cells increased from about 0.8% to about 6-7% in this one step. Then treatment of the cells with 8 hours of trypsin, suspension culture for 5 days, and then adherent culture for 5 days resulted in about 66% cells positive for CD105 and SSEA3 MUSE cells.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein M then entireties.

1. A method of enriching multi-lineage stress enduring (MUSE) cells, comprising:
providing a plurality of starting mesenchymal cells of an animal;
plating the plurality of starting mesenchymal cells on a substrate;
culturing the plurality of starting mesenchymal cells plated on the substrate in a first medium for a first period of time, wherein the first period of time is about 3-10 days; and
obtaining cells adherent to the substrate to produce a population of adherent mesenchymal cells, 3% or more of the population of adherent mesenchymal cells being MUSE cells.

2. The method of claim 1, wherein the method further comprises detaching the cells adherent to the substrate from the substrate to obtain a plurality of suspended cells.

3. The method of claim 2, wherein the method further comprises exposing the plurality of the suspended cells to trypsin in a second medium for a second period of time to obtained a plurality of trypsin-exposed cells.

4. The method of claim 3, wherein the method further comprises culturing in suspension the plurality of trypsin-exposed cells for a third period of time.

5. The method of claim 4, wherein the method further comprises, after the culturing in suspension step, culturing the plurality of trypsin-exposed cells in an adherent culture for a fourth period of time to obtain an expanded cell population, 30% or more of the expanded cell population being MUSE cells.

6. The method of claim 1, wherein the animal is a mammal.

7. The method of claim 6, wherein the mammal is a human.

8. The method of claim 1, wherein the plurality of starting cells are obtained from a tissue of the animal.

9. The method of claim 8, wherein the tissue is umbilical cord blood, bone marrow, amniotic fluid, adipose tissue, placenta, or peripheral blood.

10. The method of claim 9, wherein the tissue is umbilical cord blood.

11. The method of claim 1, wherein the starting mesenchymal cells are mononuclear cells.

12. The method of claim 1, wherein the starting mesenchymal cells are obtained from the animal by a method comprising osmotic gradient centrifugation.

13. The method of claim 1, wherein the substrate contains gelatin.

14. The method of claim 1, wherein the first medium contains serum.

15. The method of claim 1, wherein the first period of time is about 3-5 days or about 4 days.

16. The method of claim 1, further comprising removing cells that are not attached to the substrate within 12-36 hours, 18-30 hours, or 24 hours after the starting mesenchymal cells are plated on the substrate.

17. The method of claim 2, wherein the cells are detached via a non-trypsin means.

18. The method of claim 3, wherein second period of time is about 4-12 hours, 6-10 hours, or 8 hours.

19. The method of claim 3, wherein second medium is a growth medium.

20. The method of claim 4, wherein the third period of time is about 3-10 days, 4-6 days, or 5 days.

21. The method of claim 5, wherein the fourth period of time is about 3-10 days, 4-6 days, or 5 days.

22. A MUSE cell population produced according to the method of claim 1.
